(19) United States
(12) Patent Application Publication
(10) Pub. No.: US 2024/0353399 A1
(43) Pub. Date: Oct. 24, 2024

(54) MICROSPHERE UNDERGOING DIVISIONAL FUNCTIONALIZATION MODIFICATION, AND APPLICATION THEREOF

(71) Applicant: Xiamen Yijiacheng Technology Co., Ltd, Xiamen (CN)

(72) Inventor: Fangcheng XU, Xiamen (CN)

(73) Assignee: Xiamen Yijiacheng Technology Co., Ltd, Xiamen (CN)

(21) Appl. No.: 18/136,377

(22) Filed: Apr. 19, 2023

(51) Int. Cl. G01N 33/543

(52) U.S. Cl. CPC G01N 33/54326

(57) ABSTRACT

A microsphere undergoing divisional functionalization modification includes: a magnetically-modified microsphere body, where a surface of the microsphere body is provided with a plurality of annular subzones that include a capture zone, a detection zone, and a center of gravity (CG) adjustment zone sequentially from top to bottom; a surface of the capture zone is subjected to bioactive modification such that the surface has an activity to capture a biological particle or molecule; the detection zone is modified by fluorescence or a quantum dot (QD) etc. for optical detection; a surface of the CG adjustment zone is coated with a coating that has a greater specific gravity than the microsphere body; and the CG adjustment zone is configured to adjust a CG of the microsphere body to keep the capture zone facing up. The microsphere can integrate the capture, detection, and separation of one or more biological particles or molecules.

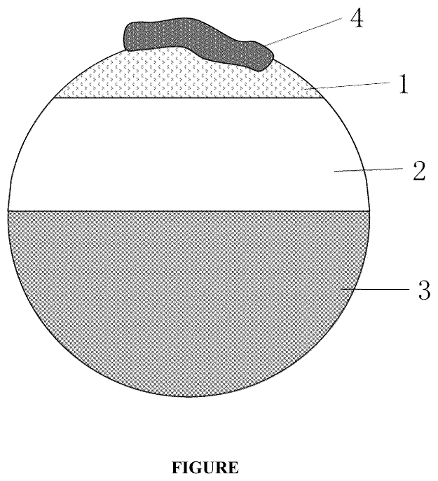

### TECHNICAL FIELD

The present disclosure relates to the technical field of biological materials, and in particular to a microsphere undergoing divisional functionalization modification, and an application thereof.

### BACKGROUND

Cells, bacteria, viruses, proteins, nucleic acids, and the like have particle characteristics. For example, cells usually have a particle size of 2 μm to 50 μm, bacteria usually have a particle size of 0.5 μm to 2 μm, viruses usually have a particle size of 15 nm to 200 nm, and proteins and nucleic acids each usually have a particle size of 0.2 nm to 2 nm. There are differences among particles even of a same organism. For example, novel coronavirus with a particle size of 100 nm can undergo substitution of 2 bases per month, resulting in differences among different particles of the original viruses. For cancer cells, at an early stage of cancerization, most cells are normal cells, and only a few cells are cancerous cells. At an advanced stage of cancerization, cancerous cells are significantly increased, and 1,000 out of 10,000 cells may be cancerous cells. In the current detection method, a large number of cells are taken as a whole for research, which can detect advanced cancer, but is hard to achieve the accurate detection of early cancer. The application of a single biological particle as a research object is an important trend in biological research, which can effectively overcome the shortcomings of existing methods.

The single particle technique includes capture, detection, and separation of a single particle, where the capture refers to the transfer of the single particle from the liquid to the separation carrier, the separation refers to the separate transfer of a carrier including the single particle, and the detection refers to the observation of a single particle capture process and the signal tracking of a single particle transfer process; and to obtain a single particle with a high throughput, these processes must be integrated together. However, because viruses mostly are very small, having a size of 15 nm to 200 nm, they are able to be observed under an electron microscope or a laser confocal fluorescence microscope, but the separation of viruses is hard. Although bacteria and cells have a larger size than viruses, in the existing technologies, single bacteria and single cells are often acquired through a single-colony technique of a petri dish or micromanipulation under an optical microscope, which has the disadvantages of low separation efficiency and unavailable separation of tiny biological single particles. Therefore, a new efficient and broad-spectrum method needs to be developed for the separation, purification, and detection of a biological particle.

### SUMMARY

To solve the above problems, the present disclosure provides a microsphere undergoing divisional functionalization modification, and an application thereof.

The present disclosure adopts the following technical solutions.

The present disclosure provides a microsphere undergoing divisional functionalization modification, including: a magnetically-modified microsphere body, where a surface of the microsphere body is provided with a plurality of annular subzones that include a capture zone, a detection zone, and a center of gravity (CG) adjustment zone sequentially from top to bottom; a surface of the capture zone is subjected to bioactive modification such that the surface has an activity to capture a biological particle or molecule; the detection zone is modified by fluorescence or a quantum dot (QD) for optical detection; a surface of the CG adjustment zone is coated with a coating that has a greater specific gravity than the microsphere body; and the CG adjustment zone is configured to adjust a CG of the microsphere body to keep the capture zone facing up.

Further, the surface of the capture zone is subjected to any bioactive modification selected from the group consisting of amination modification, carboxylation modification, hydroxylation modification, streptavidin modification, QD modification, antibody modification, and protein modification.

Further, the capture zone is configured to capture at least one biological particle or molecule, and the capture zone has a diameter of 15 nm to 45 μm.

Further, an area of the CG adjustment zone is ¼ to ¾ of a surface area of the microsphere body; and the coating of the CG adjustment zone is any one selected from the group consisting of a metal coating, a metal oxide coating, and an inorganic coating, and has a thickness of 5 nm to 100 nm.

Further, the coating of the CG adjustment zone includes any one selected from the group consisting of a platinum coating, a gold coating, a chromium coating, an aluminum oxide coating, a titanium oxide coating, and a silicon dioxide coating.

Further, the microsphere body has a diameter of 0.5 μm to 60 μm.

Further, the microsphere body is a magnetic sphere with superparamagnetism.

Further, the microsphere body is any one selected from the group consisting of a polystyrene (PS) magnetic microsphere, a silicon dioxide magnetic microsphere, and a polymethylmethacrylate (PMMA) magnetic microsphere.

An application of the microsphere undergoing divisional functionalization modification is provided, where the microsphere is configured to achieve any one or more selected from the group consisting of capture, detection, and separation of at least one biological particle or molecule.

Further, the biological particle includes any one selected from the group consisting of a virus single particle, a protein single particle, an exosome single particle, a nucleic acid single particle, a single bacterium, and a single cell.

After adopting the above technical solutions, the present disclosure has the following advantages compared with the background:
1. A surface of the microsphere of the present disclosure is subjected to divisional functionalization modification, and a surface of a microsphere body is divided into a plurality of annular subzones. A surface of a capture zone located at a top of the microsphere body is subjected to bioactive modification such that the surface has an excellent activity to capture a biological particle or molecule, and a diameter of the capture zone is controlled in a range of 15 nm to 45 μm to achieve the capture of a single or a plurality of biological particles or molecules; a detection zone located in the middle of the microsphere body is modified by fluorescence or QD for optical detection; and a surface of a CG adjustment zone located at a bottom of the microsphere body is coated with a coating that has a relatively large specific gravity, and can adjust a CG of the microsphere body to keep the capture zone facing up to form a tumbler structure, such that a biological particle or molecule captured in the capture zone can always face up, which is convenient for the capture, separation, observation, and detection of the biological particle or molecule. In addition, the microsphere body is a magnetically-modified microsphere, such that the microsphere can be easily manipulated based on the magnetic characteristics of the microsphere to achieve the separation of a single biological particle or molecule. A capture zone with a similar size to the biological particle or molecule is prepared according to a size of the biological particle or molecule to achieve the capture and separation of a single biological particle or molecule.
2. The microsphere of the present disclosure is configured to capture, detect, or separate at least one biological particle or molecule, thereby integrating the capture, detection, and separation of a single or a plurality of biological particles or molecules.

### BRIEF DESCRIPTION OF THE DRAWINGS

FIGURE is a schematic diagram illustrating a structure of the microsphere of the present disclosure.

### REFERENCE NUMERALS

1: capture zone; 2: detection zone; 3: CG adjustment zone; and 4: biological particle.

### DETAILED DESCRIPTION OF THE EMBODIMENTS

In order to make the objectives, technical solutions, and advantages of the present disclosure more clear, the present disclosure is further described in detail below with reference to the accompanying drawings and examples. It should be understood that the specific examples described herein are merely intended to explain the present disclosure, rather than to limit the present disclosure.

### Example 1

As shown in the figure, this example provides a microsphere undergoing divisional functionalization modification, including: a magnetically-modified microsphere body, where a surface of the microsphere body is provided with a plurality of annular subzones that include a capture zone 1, a detection zone 2, and a CG adjustment zone 3 sequentially from top to bottom; a surface of the capture zone 1 is subjected to bioactive modification such that the surface has an activity to capture a biological particle 4 or molecule; the detection zone 2 is modified by fluorescence for optical detection; a surface of the CG adjustment zone 3 is coated with a coating that has a greater specific gravity than the microsphere body; and the CG adjustment zone 3 is configured to adjust a CG of the microsphere body to keep the capture zone 1 facing up.

The microsphere body has a diameter of 5 μm, and the microsphere body is a silicon dioxide magnetic microsphere.

The surface of the capture zone 1 is subjected to amination bioactive modification, is configured to capture at least one biological particle or molecule, and has a diameter of 500 nm.

An area of the CG adjustment zone 3 is ½ of a surface area of the microsphere body; and the coating of the CG adjustment zone 3 is a platinum coating, and the platinum coating has a thickness of 20 nm.

### Example 2

As shown in the figure, this example provides an application of the microsphere undergoing divisional functionalization modification. The microsphere of Example 1 may be used for the capture and separation of at least one biological particle 4. The biological particle 4 includes any one selected from the group consisting of a virus single particle, a protein single particle, an exosome single particle, a nucleic acid single particle, a single bacterium, and a single cell.

In this example, the microsphere of Example 1 is used for the capture and separation of the biological particle 4, and an application method is as follows:
S1. an array of microspheres undergoing divisional functionalization modification is arranged on a chip including cylindrical micropores;
S2. a solution including a biological particle is allowed to pass through a surface of the chip, and the biological particle 4 is captured by the capture zone 1 of the microsphere;
S3. the chip is washed multiple times with phosphate buffered saline (PBS) or deionized water to remove excessive virus particles; and
S4. the microsphere capturing the biological particle 4 in the chip is taken out by ultrasonic or magnetic separation and then transferred to another chip including cylindrical micropores, thereby completing the capture and separation of the biological particle 4.

The above are merely preferred specific implementations of the present disclosure, but the protection scope of the present disclosure is not limited thereto. Any modification or replacement easily conceived by those skilled in the art within the technical scope of the present disclosure should fall within the protection scope of the present disclosure. Therefore, the protection scope of the present disclosure should be subject to the protection scope of the claims.

What is claimed is:

1. A microsphere undergoing a divisional functionalization modification, comprising: a magnetically-modified microsphere body, wherein a surface of the magnetically-modified microsphere body is provided with a plurality of annular subzones, and each of the plurality of annular subzones comprises a capture zone, a detection zone, and a center of gravity (CG) adjustment zone sequentially from top to bottom; a surface of the capture zone is subjected to a bioactive modification such that the surface of the capture zone has an activity to capture a biological particle or molecule; the detection zone is modified by fluorescence or a quantum dot (QD) for an optical detection; a surface of the CG adjustment zone is coated with a coating, and the coating has a greater specific gravity than the magnetically-modified microsphere body; and
the CG adjustment zone is configured to adjust a CG of the magnetically-modified microsphere body to keep the capture zone facing up.

2. The microsphere undergoing the divisional functionalization modification according to claim 1, wherein the surface of the capture zone is subjected to the bioactive modification selected from the group consisting of an amination modification, a carboxylation modification, a hydroxylation modification, a streptavidin modification, a QD modification, an antibody modification, and a protein modification.

3. The microsphere undergoing the divisional functionalization modification according to claim 2, wherein the capture zone is configured to capture at least one biological particle or molecule, and the capture zone has a diameter of 15 nm to 45 μm.

4. The microsphere undergoing the divisional functionalization modification according to claim 1, wherein an area of the CG adjustment zone is ¼ to ¾ of a surface area of the magnetically-modified microsphere body; and the coating of the CG adjustment zone is one selected from the group consisting of a metal coating, a metal oxide coating, and an inorganic coating, and the coating of the CG adjustment zone has a thickness of 5 nm to 100 nm.

5. The microsphere undergoing the divisional functionalization modification according to claim 4, wherein the coating of the CG adjustment zone comprises one selected from the group consisting of a platinum coating, a gold coating, a chromium coating, an aluminum oxide coating, a titanium oxide coating, and a silicon dioxide coating.

6. The microsphere undergoing the divisional functionalization modification according to claim 1, wherein the magnetically-modified microsphere body has a diameter of 0.5 μm to 60 μm.

7. The microsphere undergoing the divisional functionalization modification according to claim 6, wherein the magnetically-modified microsphere body is a magnetic sphere with superparamagnetism.

8. The microsphere undergoing the divisional functionalization modification according to claim 7, wherein the magnetically-modified microsphere body is one selected from the group consisting of a polystyrene (PS) magnetic microsphere, a silicon dioxide magnetic microsphere, and a polymethylmethacrylate (PMMA) magnetic microsphere.

9. A method of an application of the microsphere undergoing the divisional functionalization modification according to claim 1, wherein the microsphere is configured to achieve one or more selected from the group consisting of a capture, a detection, and a separation of at least one biological particle or molecule.

10. The method of the application of the microsphere undergoing the divisional functionalization modification according to claim 9, wherein the at least one biological particle comprises one selected from the group consisting of a virus single particle, a protein single particle, an exosome single particle, a nucleic acid single particle, a single bacterium, and a single cell.

11. The method of the application of the microsphere undergoing the divisional functionalization modification according to claim 9, wherein the surface of the capture zone is subjected to the bioactive modification selected from the group consisting of an amination modification, a carboxylation modification, a hydroxylation modification, a streptavidin modification, a QD modification, an antibody modification, and a protein modification.

12. The method of the application of the microsphere undergoing the divisional functionalization modification according to claim 11, wherein the capture zone is configured to capture at least one biological particle or molecule, and the capture zone has a diameter of 15 nm to 45 μm.

13. The method of the application of the microsphere undergoing the divisional functionalization modification according to claim 9, wherein an area of the CG adjustment zone is ¼ to ¾ of a surface area of the magnetically-modified microsphere body; and the coating of the CG adjustment zone is one selected from the group consisting of a metal coating, a metal oxide coating, and an inorganic coating, and the coating of the CG adjustment zone has a thickness of 5 nm to 100 nm.

14. The method of the application of the microsphere undergoing the divisional functionalization modification according to claim 13, wherein the coating of the CG adjustment zone comprises one selected from the group consisting of a platinum coating, a gold coating, a chromium coating, an aluminum oxide coating, a titanium oxide coating, and a silicon dioxide coating.

15. The method of the application of the microsphere undergoing the divisional functionalization modification according to claim 9, wherein the magnetically-modified microsphere body has a diameter of 0.5 μm to 60 μm.

16. The method of the application of the microsphere undergoing the divisional functionalization modification according to claim 15, wherein the magnetically-modified microsphere body is a magnetic sphere with superparamagnetism.

17. The method of the application of the microsphere undergoing the divisional functionalization modification according to claim 16, wherein the magnetically-modified microsphere body is one selected from the group consisting of a polystyrene (PS) magnetic microsphere, a silicon dioxide magnetic microsphere, and a polymethylmethacrylate (PMMA) magnetic microsphere.
